(19) United States
(12) Patent Application Publication
(10) Pub. No.: US 2023/0000331 A1
(43) Pub. Date: Jan. 5, 2023

(54) ENDOSCOPE SYSTEM, ENDOSCOPE, AND DISTANCE CALCULATION METHOD

(71) Applicant: OLYMPUS CORPORATION, Tokyo (JP)

(72) Inventor: Yasuo SASAKI, Tokyo (JP)

(73) Assignee: OLYMPUS CORPORATION, Tokyo (JP)

(21) Appl. No.: 17/940,153

(22) Filed: Sep. 8, 2022

Related U.S. Application Data
(63) Continuation of application No. PCT/JP2020/010203, filed on Mar. 10, 2020.

(51) Int. Cl. A61B 1/06; A61B 1/00

(52) U.S. Cl. CPC A61B 1/0638; A61B 1/00009; A61B 1/00045

(57) ABSTRACT

An endoscope system includes a light source that emits lights with first to n-th wavelengths, a lens that makes the lights with the first to n-th wavelengths parallel lights, a diffractive optical element (DOE) that converges components of the lights with the first to n-th wavelengths, the components being included in the parallel lights, into first to n-th linear lights at mutually different positions, a slit that projects, onto a subject, first to n-th pattern lights based on the first to n-th linear lights, an imager that captures, as one-frame image, an image of the subject onto which the first to n-th pattern lights are projected, and a processor being configured to calculate a distance to the subject or a shape of the subject based on the image captured by the imager.

### CROSS REFERENCE TO RELATED APPLICATION

This application is a continuation of International Patent Application No. PCT/JP2020/010203, having an international filing date of Mar. 10, 2020, which designated the United States, the entirety of which is incorporated herein by reference.

### BACKGROUND

There is a case where a size of a lesion or the like is wanted to be measured in an endoscope. To accurately measure the size, it is necessary to measure a distance from a scope to the lesion. Distance-measurement has been conventionally performed in three-dimensional measurement of a subject. As a system for the distance-measurement, a parallax system, a Time of Flight (ToF) system, and a structured light system have been known. As points of view for evaluating whether these distance-measurement systems are appropriate, consideration will be given to real-time measurement, real-time processing, and a reduction in diameter. The real-time measurement is a point of view indicating whether measurement is completed in a short period of time to perform distance-measurement of a moving target such as the living body with high accuracy. The real-time processing is a point of view indicating whether distance-measurement calculation can be performed in a short period of time to present information in real time during observation of a subject. The reduction in diameter is a point of view indicating whether a diameter of the leading end of a scope is not too large to mount a distance-measurement mechanism into the leading end of the scope.

The parallax system is also called stereo vision, and a parallax image is acquired by two imaging systems. The parallax system enables acquisition of the parallax image in one frame, and thereby enables the real-time measurement.

In the ToF system, measured is time in which a reflected wave of a light reaches an image sensor. The ToF system enables distance-measurement in one frame, and thereby enables the real-time measurement. Since process load for converting time to a distance is low, the ToF system enables the real-time processing.

In the structured light system, a plurality of pattern lights with mutually different phases is projected onto the subject, and an image of the subject is captured. Since processing load for converting a manner in which each pattern light is reflected to a distance is low, the structured light system enables the real-time processing. Since a mechanism for projecting pattern lights has a small size, the structured light system can reduce a diameter more than the other distance-measurement systems. For example, the specification of United States Patent Application Publication No. 2009/0225321 discloses inclusion of three light sources and a grating, and a distance-measurement method of sequentially turning on the light sources one by one, sequentially projecting three pattern lights whose phases are mutually different, capturing an image of a subject onto which each pattern light is projected, thereby acquiring three images, and calculating a distance from the three images.

### SUMMARY

In accordance with one of some aspect, there is provided an endoscope system comprising:

a light source that emits lights with first to n-th wavelengths;

a lens that makes the lights with the first to n-th wavelengths parallel lights;

a diffractive optical element (DOE) that converges components of the lights with the first to n-th wavelengths, the components being included in the parallel lights, into first to n-th linear lights at mutually different positions;

a slit that projects, onto a subject, first to n-th pattern lights based on the first to n-th linear lights;

an imager that captures, as an one-frame image, an image of the subject onto which the first to n-th pattern lights are projected; and

a processor being configured to calculate a distance to the subject or a shape of the subject based on the image captured by the imager.

In accordance with one of some aspect, there is provided an endoscope comprising:

a lens that makes lights with first to n-th wavelengths parallel lights;

a diffractive optical element (DOE) that converges components of the lights with the first to n-th wavelengths, the components being included in the parallel lights, into first to n-th linear lights at mutually different positions;

a slit that projects, onto a subject, first to n-th pattern lights based on the first to n-th linear lights; and

an imager that captures, as one-frame image, an image of the subject onto which the first to n-th pattern lights are projected.

In accordance with one of some aspect, there is provided a distance calculation method comprising:

a light source emitting lights with first to n-th wavelengths;

a lens making the lights with the first to n-th wavelengths parallel lights;

a diffractive optical element (DOE) converging components of the lights with the first to n-th wavelengths, the components being included in the parallel lights, into first to n-th linear lights at mutually different positions;

a slit projecting, onto a subject, first to n-th pattern lights based on the first to n-th linear lights;

an imager capturing, as one-frame image, an image of the subject onto which the first to n-th pattern lights are projected; and

a processor calculating a distance to the subject or a shape of the subject based on the image captured by the imager.

### BRIEF DESCRIPTION OF THE DRAWINGS

FIG. 1 illustrates a configuration example of an endoscope system.

FIG. 2 is a diagram for describing a first operation example of the endoscope system.

FIG. 3 is a diagram for describing a second operation example of the endoscope system.

FIG. 4 is a diagram for describing a wavelength of a pattern light.

FIG. 5 illustrates an example of spectral characteristics of an image sensor included in an imaging section.

FIG. 6 illustrates a first detailed configuration example of the endoscope system.

FIG. 7 illustrates a second detailed configuration example of the endoscope system.

FIG. 8 illustrates a first detailed configuration example of a pattern light projection section.

FIG. 9 illustrates a second detailed configuration example of the pattern light projection section.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected or coupled to each other with one or more other intervening elements in between.

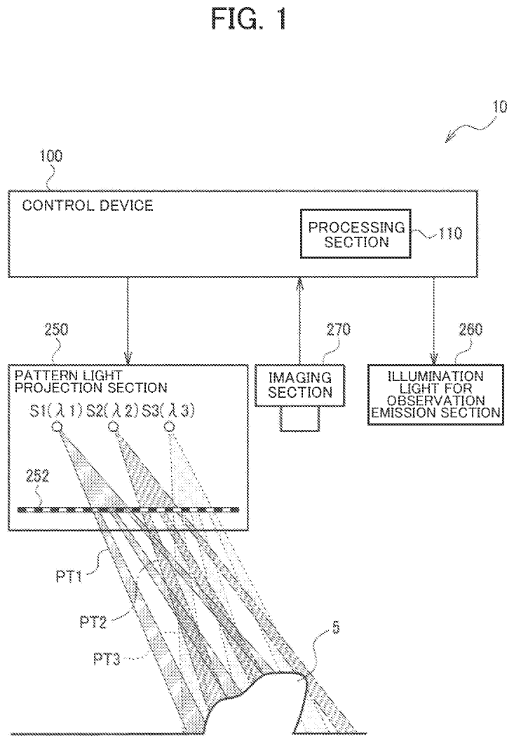

FIG. 1 illustrates a configuration example of an endoscope system 10. The endoscope system 10 includes a pattern light projection section 250, an imaging section 270, a processing section 110, and an illumination light for observation emission section 260. FIG. 1 illustrates a case where the endoscope system 10 includes a control device 100, and the processing section 110 that performs distance-measurement processing is included in the control device 100. However, the configuration is not limited thereto, and may be a configuration in which the processing section 110 that performs distance-measurement processing is arranged in an information processing device arranged outside the control device 100. The endoscope system 10 is, for example, a medical endoscope system, and a video scope used for the upper digestive tract or the lower digestive tract, a rigid scope used for surgery, or the like can be assumed.

The pattern light projection section 250 projects first to n-th pattern lights onto a subject 5, where n is an integer that is equal to or larger than 2, and n=3 in this case. Pattern lights PT1 to PT3 correspond to first to third pattern lights. The pattern lights PT1 to PT3 have striped or latticed patterns, and phases of the patterns and light wavelengths are mutually different. The imaging section 270 captures, as one-frame image, an image of the subject 5 onto which the pattern lights PT1 to PT3 are projected. The processing section 110 calculates a distance to the subject 5 or a shape of the subject 5 based on the one-frame image.

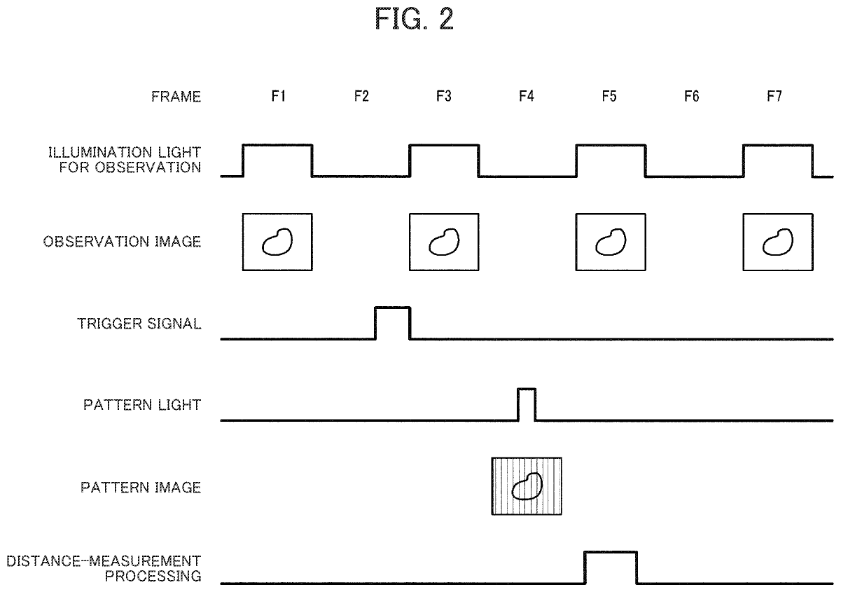

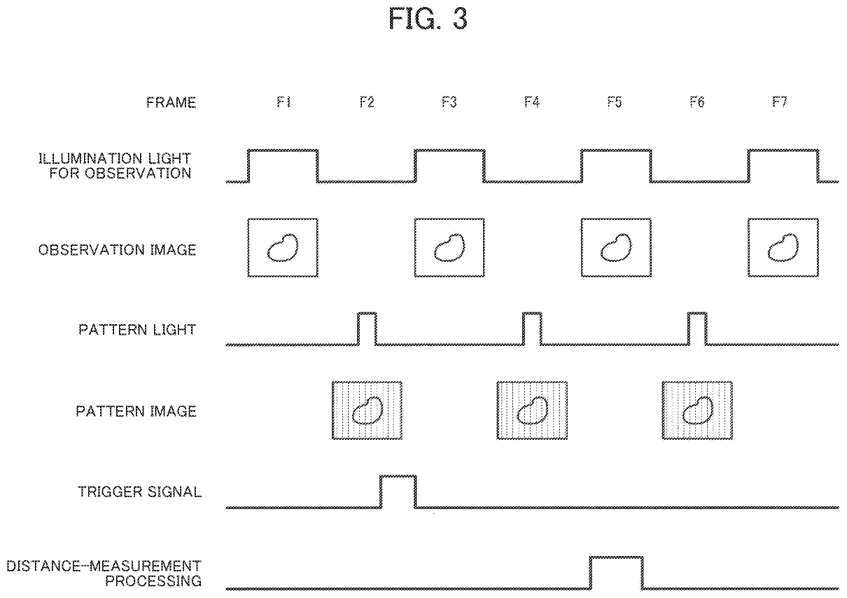

The frame mentioned herein is an exposure period to capture one image. For example, frames are periodically repeated when a movie is captured. The above-mentioned one-frame image is captured in one of these frames. For example, as described later with reference to FIGS. 2 and 3, the image of the subject 5 onto which the pattern lights PT1 to PT3 are projected is captured in a frame between frames in each of which an observation image is captured.

In accordance with the present embodiment, since the image of the subject 5 onto which the pattern lights PT1 to PT3 are projected is captured in one frame, an image necessary for distance-measurement in the structured light system can be captured in a short period of time. This enables real-time measurement in the structured light system, and thereby enables distance-measurement of a moving target such as the living body with high accuracy. Since the pattern lights PT1 to PT3 have mutually different wavelengths, a subject image captured when the pattern lights PT1 to PT3 are projected is separated from the one-frame image utilizing the difference in wavelength, and a distance can be calculated from this information.

Note that the parallax system puts high load for parallax calculation, has difficulty in performing real-time processing, and requires two imaging systems. Thus, it is difficult to reduce a diameter. In the conventional structured light system, since one frame image is captured with respect to single-time pattern projection, a plurality of frame images needs to be captured for capturing of images for all pattern projection. In this manner, it is preferable that the structured light system be adopted in the point of view of the reduction in the diameter, which is important in the endoscope. In the conventional structured light system, however, the real-time measurement is difficult because a plurality of images is captured, and there is an issue that the conventional structured light system is not suitable for distance-measurement of the moving target such as the living body with high accuracy. The present embodiment enables real-time measurement as described above, and thereby enables distance-measurement of the moving target such as the living body with high accuracy.

The endoscope system 10 may perform diagnosis support using artificial intelligence (AI). In this case, inputting information of the distance to the subject 5 or the shape of the subject 5 together with the observation image to the AI can increase accuracy of diagnosis support.

The shape obtained by distance-measurement is important as evidence when a diagnosis is made about whether or not the region of interest is a lesion. For example, in a case where a polyp is detected, measurement of a size of the polyp presents important evidence in a diagnosis about whether or not the polyp is cancer.

The configuration example illustrated in FIG. 1 is described in detail below. The pattern light projection section 250 includes first to third light sources S1 to S3 that emit lights with first to third wavelengths λ1 to λ3, respectively, and a slit section 252 in which a plurality of slits is arranged. The pattern light projection section 250 is also referred to as a pattern light projection device. As described above, pattern lights PT1 to PT3 have striped or lattice patterns.

The striped pattern is a pattern in which parallel lines are repeated in a periodic or substantially periodic manner In a case where the pattern lights PT1 to PT3 have the striped patterns, a plurality of linear slits is arranged in the slit section 252. The linear slits are parallel to each other, and arrayed in a direction orthogonal to the linear slits.

The lattice pattern is a pattern in which a first line group and a second line group are orthogonal to each other, and, in each line group, parallel lines are repeated in a periodic or substantially periodic manner In a case where the pattern lights PT1 to PT3 have the lattice patterns, lattice-shaped slits are arranged in the slit section 252. That is, a plurality of first linear slits and a plurality of second linear slits orthogonal to the first linear slits are arranged in the slit section 252. The slit section 252 is also referred to as a grating. In addition, the slit section 252 is a plate-like member provided with slits, and is also referred to as a slit plate.

The light sources S1 to S3 emit lights whose spectral peak wavelengths are wavelengths λ1 to λ3, respectively. The light sources S1 to S3 emit lights with line widths that allow respective spectra to be sufficiently separated from each other, and eject lights with line widths of, for example, several nanometers to several tens of nanometers. The light sources S1 to S3 are, as described later in a second embodiment, virtual light sources produced by using a laser diode, a diffractive optical element (DOE), and the like. Alternatively, each of the light sources S1 to S3 may be made of a light-emitting element such as a light-emitting diode and a band-pass filter. In a case where the pattern lights PT1 to PT3 have the striped patterns, each of the light sources S1 to S3 is a linear light source that is parallel to the liner slits. The light sources S1 to S3 are arrayed in a plane that is parallel to a flat surface of the slit section 252 in a direction that is identical to a direction in which the linear slits are arrayed. In a case where the pattern lights PT1 to PT3 have the lattice patterns, the light sources S1 to S3 are point-like light sources, and are arranged at different positions in the plane that is parallel to the flat surface of the slit section 252.

Lights from the light sources S1 to S3 pass through the slits of the slit section 252, whereby the pattern lights PT1 to PT3 are generated. When the pattern lights PT1 to PT3 are projected onto a flat subject that is parallel to the flat surface of the slit section 252, the pattern lights PT1 to PT3 have the striped or lattice patterns, and phases of stripes or lattices are different from each other. Taking striped pattern lights as an example, assuming that one period of stripes corresponds to a phase of 360 degrees, stripes of the pattern lights PT1 to PT3 are at ph1to ph3 degrees with respect to a position of 0 degrees serving as a reference, and the ph1 to ph3 degrees have mutually different values. Since the positions of the light sources S1 to S3 are different from each other, a phase relationship among the pattern lights PT1 to PT3 varies depending on a distance to the subject. However, in a case where the light sources S1 to S3 are arranged sufficiently close to each other, the phase relationship can be regarded as constant regardless of the distance to the subject.

The illumination light for observation emission section 260 emits an illumination light for observation used for capturing the observation image toward the subject 5. The observation image is an image for a user to observe the subject 5. In terms of comparison with a light and an image for distance-measurement, the illumination light for observation is also referred to as a normal light, and the observation image is also referred to as a normal image. The illumination light for observation is only required to be an illumination light having spectral characteristics in accordance with an observation purpose, and is, for example, a white light or a special light. Examples of the special light include an illuminating light for narrow band imaging (NBI) including a green narrow band light and a blue narrow band light. Note that the illumination light for observation emission section 260 is also referred to as an illumination light for observation emission device.

The imaging section 270 includes an objective lens that forms an image of the subject 5, and an image sensor that captures the image of the subject 5 formed by the objective lens. Either the pattern light projection section 250 or the illumination light for observation emission section 260 emits a light. When the pattern light projection section 250 emits a light, the imaging section 270 captures the image of the subject 5 onto which the pattern lights PT1 to PT3 are projected. When the illumination light for observation emission section 260 emits a light, the imaging section 270 captures the observation image. The imaging section 270 includes one image sensor, and this common image sensor captures the image with the illumination light for observation and the image with the pattern light.

The control device 100 is a device that performs control of the endoscope system 10, image processing, and the like. A scope is connected to the control device 100, and is provided with the pattern light projection section 250, the illumination light for observation emission section 260, and the imaging section 270. The control device 100 includes the processing section 110.

The processing section 110 is implemented by a circuit device in which a plurality of circuit components is mounted on a substrate. Alternatively, the processing section 110 may be an integrated circuit device such as a processor, an application-specific integrated circuit (ASIC), and a field-programmable gate array (FPGA) circuit. The processor is a central processing unit (CPU), a microcomputer, a digital signal processor (DSP), or the like. In a case where the processing section 110 is the processor, the processor executes a program in which operations of the processing section 110 are described, and thereby implements the operations of the processing section 110. The program is, for example, stored in a memory, which is not illustrated. Note that the processing section 110 is also referred to as a processing circuit or a processing device.

When the pattern lights PT1 to PT3 are projected, the processing section 110 calculates, based on an image captured by the imaging section 270, phases at respective positions of the image, and calculates, based on the phases, distances to the subject 5 at the respective positions of the image. This distance information is information such as a Z map in which a distance with respect to each pixel is calculated, and indicates a three-dimensional shape of the subject 5. The processing section 110 calculates the shape of the subject 5 from the calculated distance. Various kinds of information of the calculated shape can be assumed. Examples of the information include a length, width, longer diameter, shorter diameter, height, or depth of the region of interest, a contour of the region of interest, or a combination of some of them.

Taking the length of the region of interest as an example, a description will be given of a method of obtaining a length in a physical space from a length on the image. The processing section 110 obtains the length of the region of interest in the physical space based on the length of the region of interest on the image and the distance to the region of interest. That is, an expected angle of the region of interest viewed from the imaging section 270 can be found from an angle of view of the imaging section 270 and the length of the region of interest on the image. The processing section 110 obtains the length of the region of interest in the physical space from the expected angle and the distance to the region of interest. A value obtained by multiplying the expected angle by the distance to the region of interest is approximately equal to the length of the region of interest in the physical space.

The processing section 110 may perform gradient correction when calculating the shape of the region of interest. That is, the processing section 110 calculates a gradient of the region of interest from a distance in the periphery of the region of interest, performs gradient correction on the length or the like calculated on the image, thereby converts the length into a length or the like when the region of interest is at a correct position with respect to the imaging section 270, and outputs information of the corrected length or the like as shape information of the subject 5.

The processing section 110 may obtain not only the shape of the region of interest, but also a distance between two parts. For example, in a large intestine endoscope, it is assumed that a distance between a polyp and the anus is measured. In a case where the two parts are separate from each other and not seen within one image, a path therebetween is divided and then distances are measured. That is, the processing section 110 acquires a plurality of pattern images on the path between the two parts and connects distances calculated from the respective pattern images to calculate the distance between the two parts. The distance between a lesion and the anus serves as a material for determining whether or not functional preservation surgery should be applied.

In the present embodiment, the endoscope system 10 switches the pattern lights PT1 to PT3 and the illumination light for observation to be emitted toward the subject 5, and acquires the image with the pattern lights PT1 to PT3 and the observation image in units of one frame. Two examples of this operation are described below. The image captured when the pattern lights PT1 to PT3 are projected is hereinafter referred to as a pattern image.

FIG. 2 is a diagram for describing a first operation example of the endoscope system 10. The illumination light for observation emission section 260 emits illumination lights for observation in, among consecutive frames of F1 to F7, F1, F3, F5, and F7. In FIG. 2, a high level of a waveform indicates turning-on of a light, and a low level thereof indicates turning-off of a light. The imaging section 270 captures images in the frames F1, F3, F5, and F7 in which the illumination lights for observation are emitted. Each of the images serves as the observation image.

The pattern light projection section 250 projects the pattern lights PT1 to PT3 in a frame in which no illumination light for observation is emitted. In FIG. 2, for example, the pattern light projection section 250 projects the pattern lights PT1 to PT3 in the frame F4 after a trigger signal is input, and the imaging section 270 captures the pattern image. In FIG. 2, the high level of the waveform indicates projection of the pattern lights PT1 to PT3, and the low level thereof indicates that the pattern lights PT1 to PT3 are turned off. Time during which the pattern lights PT1 to PT3 are projected is freely determined, but is preferably short in terms of distance-measurement accuracy. The time may be set, for example, based on luminance of the pattern lights PT1 to PT3, necessary distance-measurement accuracy, and the like. The processing section 110 performs distance-measurement processing based on the pattern image captured in the frame F4. In FIG. 2, the high level of the waveform indicates execution of the distance-measurement processing. In the endoscope, there is a case where accumulative time for acquiring observation images is wanted to be made as long as possible due to an issue of lack of a light quantity of the light source for observation. In this case, a conceivable method is to acquire the observation image assuming two fames in FIG. 2 to be one frame, stop acquisition of the observation image only when the trigger signal is input, and emit pattern lights to acquire the pattern image. In this case, it is necessary to exercise ingenuity such as display of an observation image in a previous frame at the time of display of the observation image.

The trigger signal is input to the processing section 110 by, for example, a user operation. For example, a button for instructing distance-measurement is arranged on a scope operating section of the scope, and the trigger signal is input from the scope operating section to the processing section 110 when the button is pressed. Alternatively, the trigger signal may be generated inside the processing section 110. For example, the processing section 110 determines whether or not the region of interest is present in the observation image, and generates the trigger signal when detecting the region of interest in the observation image. The region of interest is, for example, a lesion such as cancer and a polyp. The processing section 110 performs AI processing or the like to detect the region of interest, and generates the trigger signal. In addition, the processing section 110 may use a result of distance-measurement of the region of interest detected by the AI processing to further perform AI processing, and thereby increase accuracy of determining the region of interest. For example, the AI processing uses the size, shape, or the like of the region of interest obtained by the distance-measurement.

FIG. 3 is a diagram for describing a second operation example of the endoscope system 10. The observation image is captured in each of the frames F1, F3, F5, and F7 similarly to FIG. 2. In FIG. 3, in all of the frames F2, F4, and F6 in which no illumination light for observation is emitted, the pattern light projection section 250 projects, regardless of the trigger signal, the pattern lights PT1 to PT3, and the imaging section 270 captures the pattern image. The processing section 110 then performs the distance-measurement processing when the trigger signal is input. That is, the processing section 110 performs the distance-measurement processing based on the pattern image captured in the frame F4 after the trigger signal is input. In this operation example, recording the pattern image in each frame enables execution of the distance-measurement processing afterwards also in frames that have not been subjected to the distance-measurement during observation.

As described with reference to FIGS. 2 and 3, the pattern light projection section 250 simultaneously projects the pattern lights PT1 to PT3 onto the subject 5 in the present embodiment. “Simultaneously” means that there is at least a timing at which all the pattern lights PT1 to PT3 are projected. Periods for projecting the pattern lights PT1 to PT3 are not necessarily matched with each other, but it is more preferable that the periods for projection be matched with each other.

The pattern lights PT1 to PT3 are simultaneously projected in accordance with the present embodiment. Thus, the pattern lights PT1 to PT3 are projected without time difference in comparison with a method of capturing an image on a frame-by-frame basis with respect to each pattern light. This enables simultaneous obtaining of the pattern images of the moving subject such as the living body image using three pattern lights, and thereby enables distance-measurement with high accuracy.

In accordance with the present embodiment, in a first frame, the illumination light for observation emission section 260 emits the illumination light for observation toward the subject 5, and the imaging section 270 captures the observation image. In a second frame that is different from the first frame, the pattern light projection section 250 projects the pattern lights PT1 to PT3 toward the subject 5, and the imaging section 270 captures the pattern image. The first frame corresponds to any one of F1, F3, F5, and F7 illustrated in FIGS. 2 and 3. The second frame corresponds to F4 in FIG. 2, or any one of F2, F4, and F6 illustrated in FIG. 3.

In accordance with the present embodiment, it is possible to capture the observation image and present the observation image to a user while performing the distance-measurement in the background, and present, to the user, information of the distance or shape obtained by the distance-measurement together with the observation image.

### 2. Wavelength of Light Source and Distance-Measurement Processing

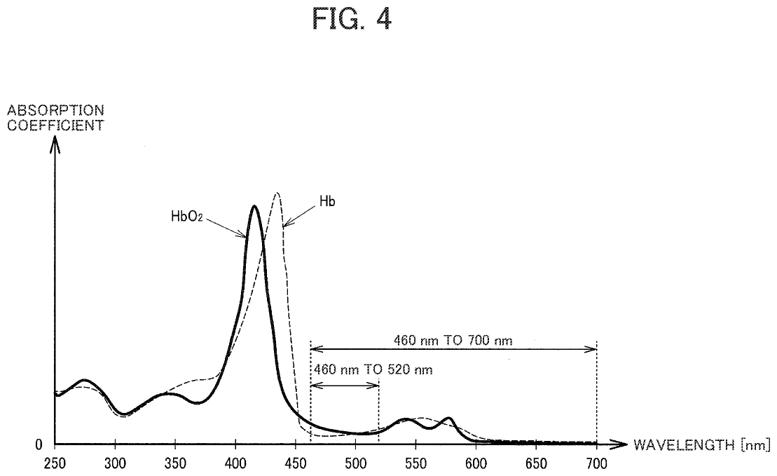

FIG. 4 is a diagram for describing wavelengths λ1 to λ3 of the pattern lights PT1 to PT3. FIG. 4 illustrates spectral characteristics of hemoglobin Hb and oxygenated hemoglobin HbO₂. Note that each of hemoglobin Hb and oxygenated hemoglobin HbO₂ is hereinafter simply referred to as hemoglobin.

An observation target in a medical endoscope is the living body, but spectral characteristics of the living body are determined mainly by spectral characteristics of hemoglobin. Hence, in the present embodiment, the wavelengths λ1 to λ3 of the pattern lights PT1 to PT3 are set based on spectral characteristics of hemoglobin.

The conventional structured light method uses a single color light. This is to equalize reflectances of respective patterns regardless of spectral characteristics of the subject. In a case where the mutually different wavelengths λ1 to λ3 are used like the present embodiment, it is preferable that reflectances of the subject with the respective wavelengths be identical. Thus, the present embodiment uses a wavelength region in which an absorption coefficient is as flat as possible in spectral characteristics of hemoglobin. That is, the wavelengths λ1 to λ3 are set so as to avoid a large absorption peak in a region of 450 nm or less and its periphery region in which a change in absorption coefficient is large.

Specifically, the wavelengths λ1 to λ3 of the pattern lights PT1 to PT3 belong to a range of 460 nm or more and 700 nm or less. Since there is a small change in spectral characteristics of hemoglobin in the range of 460 nm or more and 700 nm or less, reflectances of respective patterns are almost identical. It is more preferable that the wavelengths λ1 to λ3 of the pattern lights PT1 to PT3 belong to a range of 460 nm or more and 520 nm or less. In the range of 460 nm or more and 520 nm or less, there is a smaller change in spectral characteristics of hemoglobin than that in the range of 460 nm or more and 700 nm or less.

A mucosa serving as a target of the medical endoscope has multitudes of capillaries around its surface. As illustrated in FIG. 4, since absorption by hemoglobin is large in a wavelength region of 460 nm or less, a return light with a wavelength of 460 nm or less is extremely weak at a position where capillaries are present. In the structured light method, a distance is measured from a light quantity ratio at respective points of the pattern lights PT1 to PT3. Thus, if there is a factor other than variations in intensity of lights of the patterns, that is, variations in intensity of return lights due to differences in reflectance of capillaries or the like, it is impossible to perform accurate distance-measurement. For example, in a case where one of the pattern lights PT1 to PT3 has a wavelength of 460 nm or less, the return light of the pattern light from capillaries is extremely weak in comparison with return lights of the other pattern lights. Consequently, a light quantity becomes inaccurate, and a distance at a position of capillaries cannot be measured accurately. In the present embodiment, setting the wavelengths of the pattern lights PT1 to PT3 to the range of 460 nm or more and 700 nm or less or the range of 460 nm or more and 520 nm or less makes the light quantity ratio of the return lights accurate, and thereby enables accurate distance-measurement.

Subsequently, a description will be given of the distance-measurement processing that obtains the distance from the image captured by simultaneous emission of the pattern lights PT1 to PT3. The following description will be given of an example in which λ1=520 nm, λ2=500 nm, and λ3=480 nm.

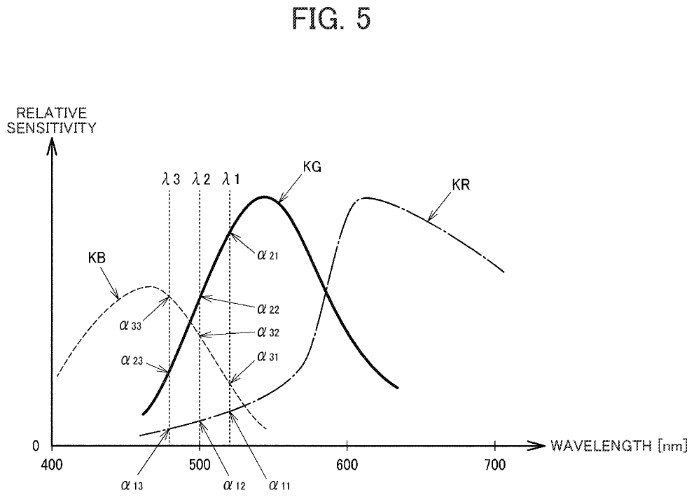

FIG. 5 illustrates an example of spectral characteristics of the image sensor included in the imaging section 270. The image sensor includes first to n-th color pixels that receive lights in first to n-th colors, respectively. Assume that n=3, and the image sensor is of a red, green, and blue (RGB) Bayer array type where R is a first color, G is a second color, and B is a third color. In FIG. 5, KR is relative sensitivity of an R pixel, KG is relative sensitivity of a G pixel, and KB is relative sensitivity of a B pixel.

As illustrated in FIG. 5, sensitivity of an i-th color pixel in a j-th wavelength λj is aᵢⱼ, where each of i and j is an integer that is equal to or larger than 1 and equal to or smaller than n. The processing section 110 extracts images of the subject 5 when the respective pattern lights are projected based on the sensitivity aᵢⱼ intensity values p₁, p₂, and p₃ of R, G, and B in the pattern image. The processing section 110 then calculates the distance to the subject 5 or the shape of the subject 5 from phases based on the images of the subject 5 when the respective pattern lights are projected. Details of the processing will be described below.

First, a relationship represented by the following Expression (1) holds between the intensity values p₁, p₂, and p₃ that can be acquired from the pattern image and the images of the subject 5 when the respective pattern lights to be obtained are projected. In this expression, q₁ is an intensity value in the image of the subject 5 when the pattern light PT1 with the wavelength λ1 is projected. Similarly, q₂ and q₃ are intensity values in the respective images of the subject 5 when the pattern light PT2 with the wavelength λ2 and the pattern light PT3 with the wavelength λ3 are projected. Assume that a position in the pattern image is represented by (x, y). The position (x, y) is, for example, pixel coordinates. In the following Expression (1), q₁, q₂, and q₃ on the left-hand side and p₁, p₂, and p₃ on the right-hand side are intensity values regarding the identical position (x, y).

$$[ Expression 1 ]$$

$$( \begin{matrix}{p}_{1} \\ {p}_{2} \\ {p}_{3}\end{matrix} ) = ( \begin{matrix}{α}_{11} & {α}_{12} & {α}_{13} \\ {α}_{21} & {α}_{22} & {α}_{23} \\ {α}_{31} & {α}_{32} & {α}_{33}\end{matrix} ) ( \begin{matrix}{q}_{1} \\ {q}_{2} \\ {q}_{3}\end{matrix} ) \qquad ( 1 )$$

Assume that a matrix whose element is aᵢⱼ is A. The wavelengths λ1 to λ3 are selected so that each row vector of the matrix A is a linearly independent vector, that is, (a₁₁, a₁₂, a₁₃), (a₂₁, a₂₂, a₂₃), and (a₃₁, a₃₂, a₃₃) are linearly independent vectors. Accordingly, since the matrix A has an inverse, the above-mentioned Expression (1) can be modified to the following Expression (2). The processing section 110 uses the following Expression (2) to calculate the intensity values q₁, q₂, and q₃ at each (x, y).

$$[ Expression 2 ]$$

$$( \begin{matrix}{q}_{1} \\ {q}_{2} \\ {q}_{3}\end{matrix} ) = {( \begin{matrix}{α}_{11} & {α}_{12} & {α}_{13} \\ {α}_{21} & {α}_{22} & {α}_{23} \\ {α}_{31} & {α}_{32} & {α}_{33}\end{matrix} )}^{- 1} ( \begin{matrix}{p}_{1} \\ {p}_{2} \\ {p}_{3}\end{matrix} ) \qquad ( 2 )$$

Note that the following Expressions (3) and (4) correspond to the above-mentioned Expressions (1) and (2) that are rewritten to another description format, but mean the same. Aᵢⱼ represents an ij component of the matrix A.

[Expression 3]

pᵢ=Aᵢⱼqⱼ

Aᵢⱼ=αᵢⱼ (3)

[Expression 4]

qⱼ=(A⁻¹)ⱼᵢpᵢ (4)

The processing section 110 uses a look up table (LUT) to convert the intensity values q₁, q₂, and q₃ to a phase WPh, as indicated by the following Expression (5). The LUT is a table in which a combination of the intensity values q₁, q₂, and q₃ and the phase WPh, and is preliminarily stored in a memory or the like in the control device 100. The phase WPh is a wrapped phase, and the processing section 110 performs unwrapping processing on the phase WPh and obtains the distance from a phase after the unwrapping processing. The unwrapping processing is processing of connecting phases that are discontinuous at a boundary in a period of stripes to convert the phases into continuous phases. That is, in the wrapped phase, a phase of one stripe belongs to 0 to 360 degrees, and a phase of a stripe next to the one stripe also belongs to 0 to 360 degrees. The unwrapping processing is to connect these phases at 0 to 720 degrees.

$$\begin{matrix}[ Expression 2 ] & \\ ( \begin{matrix}{q}_{1} \\ {q}_{2} \\ {q}_{3}\end{matrix} ) = {( \begin{matrix}{α}_{11} & {α}_{12} & {α}_{13} \\ {α}_{21} & {α}_{22} & {α}_{23} \\ {α}_{31} & {α}_{32} & {α}_{33}\end{matrix} )}^{- 1} ( \begin{matrix}{p}_{1} \\ {p}_{2} \\ {p}_{3}\end{matrix} ) & ( 2 )\end{matrix}$$

Assuming that a reference plane is set at a reference distance, a phase of a pattern light on the reference plane is determined. A difference between the phase serving as a reference and the phase obtained by the above-mentioned processing represents a relative distance between the reference plane and the subject 5. That is, the processing section 110 calculates the distance to the subject 5 from the difference between the phase serving as the predetermined reference and the phase obtained by the above-mentioned processing.

In a case where positions of the light sources S1 to S3 can be approximated to be sufficiently close to each other, the phase WPh may be obtained by functional calculation as indicated by the following Expression (6). When an argument is v/u, arctan 2 is a function for obtaining an angle of deviation of a point (u, v) in a uv orthogonal coordinates. An argument u of arctan 2 may be a negative value, and a value range is −π to +π.

$$[ Expression 6 ]$$

$$Wph ( x , y ) = arctan 2 ( \frac{\sqrt{3} ⨯ ( {q}_{1} ( x , y ) - {q}_{3} ( x , y ) )}{2 ⨯ {q}_{2} ( x , y ) - {q}_{1} ( x , y ) - {q}_{3} ( x , y )} ) \qquad ( 6 )$$

The present embodiment enables determination of a phase from one-frame image obtained by simultaneous projection of the pattern lights PT1 to PT3, and enables calculation of the distance to the subject 5 using the phase. Additionally, using a whole-space tabulation method enables measurement of the distance without conversion of a ratio of the pattern lights PT1 to PT3 at each point to a phase.

### 3. Detailed Configuration Example

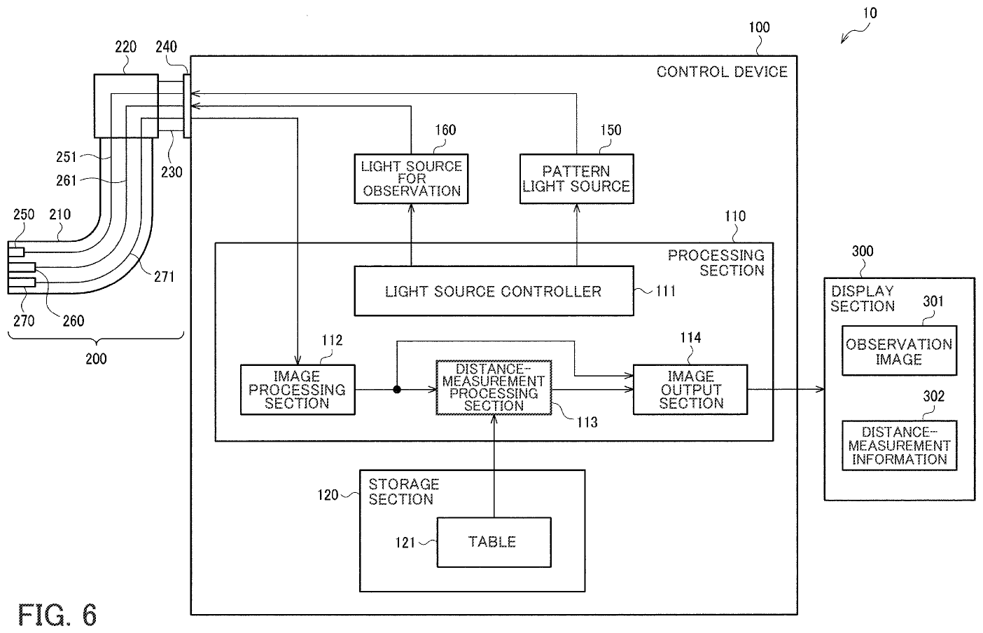

FIG. 6 illustrates a first detailed configuration example of the endoscope system 10. The endoscope system 10 includes a scope 200, the control device 100, and a display section 300. Note that a constituent element that has been already described with reference to FIG. 1 or the like is denoted by an identical reference sign, and a description thereof is omitted as appropriate.

The scope 200 includes a flexible section 210 that is inserted into the living body, an operating section 220, a connector 240 that connects the scope 200 to the control device 100, and a universal code 230 that connects the operating section 220 and the connector 240 to each other.

The pattern light projection section 250, the illumination light for observation emission section 260, and the imaging section 270 are arranged at the leading end of the flexible section 210. An end portion on the opposite side of the leading end of the flexible section 210 is connected to the operating section 220. The operating section 220 is a device for performing an angle operation of the flexible section 210, an operation of a treatment tool, an operation for air supply and water supply, and the like. An optical fiber 251, a light guide 261, and a signal line 271 are arranged inside the flexible section 210, the operating section 220, and the universal code 230. The optical fiber 251 connects the pattern light projection section 250 and the connector 240 to each other. The light guide 261 connects the illumination light for observation emission section 260 and the connector 240 to each other. The signal line 271 connects the imaging section 270 and the connector 240 to each other. The optical fiber 251, the light guide 261, and the signal line 271 are connected to an optical fiber, a light guide, and a signal line inside the control device 100, respectively, by the connector 240.

The control device 100 includes the processing section 110, a storage section 120, a pattern light source 150, and a light source for observation 160.

The light source for observation 160 is a light source that generates an illumination light for observation. The light source for observation 160 includes a white light source and an optical system that causes a light emitted from the white light source to be incident on the light guide. The white light source is, for example, a xenon lamp or a white light emitting diode (LED).

The pattern light source 150 is a light source that emits laser lights with the wavelengths λ1 to λ3. The pattern light source 150 includes first to third laser diodes that generate laser lights with the wavelengths λ1 to λ3, and an optical system that causes the laser lights emitted from the first to third laser diodes to be incident on the optical fiber.

The storage section 120 is a storage device such as a memory and a hard disk drive. The memory is a semiconductor memory, and a volatile memory such as a random-access memory (RAM), or a nonvolatile memory such as an electrically erasable programmable read-only memory (EEPROM). The storage section 120 stores a program, data, and the like necessary for an operation of the processing section 110. In addition, the storage section 120 stores the LUT described above with reference to the Expression (5) as a table 121. In a case where the phase is obtained by functional calculation like the Expression (6), the table 121 may be omitted.

The processing section 110 includes a light source controller 111, an image processing section 112, a distance-measurement processing section 113, and an image output section 114. These sections may be implemented by individual hardware circuits. Alternatively, a function of each section may be implemented by the processor implementing a program in which operations of each section is described.

The light source controller 111 controls the pattern light source 150 and the light source for observation 160. The light source controller 111 controls a light-emission timing, light emission period, and light quantity of each of the pattern light source 150 and the light source for observation 160.

The image processing section 112 performs image processing on an image signal input from the imaging section 270 via the signal line 271. The image processing section 112 performs processing of generating an RGB color image from a raw image. In addition, the image processing section 112 may perform, for example, white balance processing, gradation processing, highlighting processing, or the like. An image output from the image processing section 112 in a frame in which the illumination light for observation is emitted is the observation image, and an image output from the image processing section 112 in a frame in which the pattern lights are projected is the pattern image.

The distance-measurement processing section 113 performs the distance-measurement processing described with reference to the Expressions (1) to (6) to obtain a distance to each position of the subject from the pattern image. The distance-measurement processing section 113 obtains the shape from the distance to each point of the subject. As described above, the shape is the shorter diameter, longer diameter, width, length, height, depth, or the like of the region of interest. The information regarding the distance or the shape is hereinafter collectively referred to as distance-measurement information. Note that the distance-measurement processing section 113 may obtain the distance to each position in the entire region of the pattern image, or may obtain the distance to each position only in a partial region such as the region of interest. The distance-measurement processing section 113 may obtain, as the shape information, a length, a height, or the like between points designated by the user.

The distance-measurement processing section 113 may calculate a gradient of the region of interest based on the distance to the periphery of the region of interest, which is a target of calculation of the shape, and perform gradient correction on the shape of the region of interest based on the gradient. For example, the processing section 110 performs AI processing or the like, which will be described later, to detect the region of interest. The distance-measurement processing section 113 obtains distances to three or more points in the periphery of the region of interest, and obtains a gradient of the surface of the subject in the periphery of the region of interest based on the distances. The gradient is an angle formed between a line-of-sight of a camera of the imaging section 270 and the surface. The distance-measurement processing section 113 performs projective transformation so that the surface of the subject is at a correct position with respect to the imaging section 270, and thereby obtains the shape of the region of interest in the subject at the correct position. The shape mentioned herein is a so-called size, and is, for example, the length, the width, the longer diameter, the shorter diameter, or the like.

The image output section 114 outputs a display image to the display section 300 based on the observation image and the distance-measurement information. The display section 300 is a display such as a liquid crystal display device and an electro luminescence (EL) display device. The image output section 114 displays, for example, an observation image 301 and distance-measurement information 302 side by side in a display region of the display section 300. Alternatively, the image output section 114 may superimpose the distance-measurement information on the observation image to display the observation image and the distance-measurement information. For example, information indicating the shape of the region of interest may be superimposed on the region of interest.

Specifically, the image processing section 112 generates the observation image based on the image captured by the imaging section 270 in the first frame. The image output section 114 causes the display section 300 to display the observation image 301. The first frame corresponds to any one of F1, F3, F5, and F7 illustrated in FIG. 2 or 3. The distance-measurement processing section 113 calculates, as background processing of display of the observation image 301, the distance to the subject or the shape of the subject based on the image captured by the imaging section 270 in the first frame. The second frame corresponds to F4 in FIG. 2, or any one of F2, F4, and F6 illustrated in FIG. 3. The image output section 114 adds the distance-measurement information 302 based on the distance to the subject or the shape of the subject to the observation image 301, and causes the display section 300 to display the observation image 301.

While the pattern light source 150 is arranged in the control device 100 in FIG. 6, the pattern light source 150 may be arranged in the operating section 220 of the scope 200. While the pattern light source 150 and the light source for observation 160 are separately arranged in FIG. 6, the light source for observation 160 may serve as both thereof. In this case, an optical fiber that connects the light source for observation 160 and the illumination light for observation emission section 260 to each other baches off in the scope 200, and the branched optical fibers are connected to the pattern light projection section 250. This can omit connection between the pattern light projection section 250 and the control device 100.

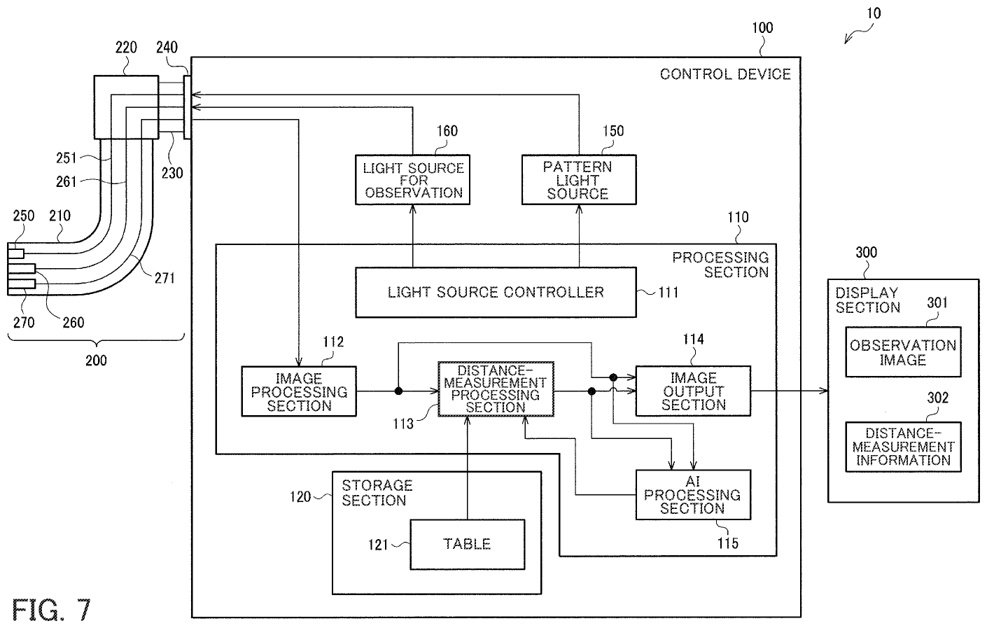

FIG. 7 illustrates a second detailed configuration example of the endoscope system 10. In FIG. 7, the processing section 110 further includes an AI processing section 115. Note that a constituent element that has been already described with reference to FIGS. 1 and 6 or the like is denoted by an identical reference sign, and a description thereof is omitted as appropriate.

The image processing section 112 generates the observation image based on an image captured by the imaging section 270 in the first frame. The first frame corresponds to any one of F1, F3, F5, and F7 illustrated in FIG. 2 or 3. The distance-measurement processing section 113 calculates the distance to the subject or the shape of the subject based on an image captured by the imaging section in the second frame. The second frame corresponds to F4 in FIG. 2, or any one of F2, F4, and F6 illustrated in FIG. 3. The AI processing section 115 performs AI processing based on the observation image and the distance to the subject or the shape of the subject to make determination regarding detection of presence of the region of interest or discrimination of a state. The detection of the presence of the region of interest is detection of whether or not the region of interest serving as a detection target is present in an image. The discrimination of the state of the region of interest is discrimination of a classification category indicating the state of the region of interest. Examples of the classification category include a lesion type such as cancer and a polyp, an index indicating a degree of progress such as a stage of cancer.

A more detailed description will be given with reference to FIG. 2. The observation image captured in the frame F1 is input to the AI processing section 115, and the AI processing section 115 performs AI processing based on the observation image to detect the region of interest. When detecting the region of interest, the AI processing section 115 outputs a trigger signal to the distance-measurement processing section 113. The distance-measurement processing section 113 calculates, based on an image captured in the frame F4 after the trigger signal is input, the distance to the subject or the shape of the subject, and inputs the distance to the subject or the shape of the subject to the AI processing section 115. The observation image captured in the frame F3, the frame F5, or the like is input to the AI processing section 115. The AI processing section 115 makes determination regarding the detection of the region of interest or the discrimination of the state based on the observation image and the distance to the subject or the shape of the subject. A result of this second determination may be used for generating a trigger signal again. Alternatively, the result of the second determination may be output to the image output section 130, and the image output section 130 may add the result of determination to the observation image and cause the display section 300 to display the observation image.

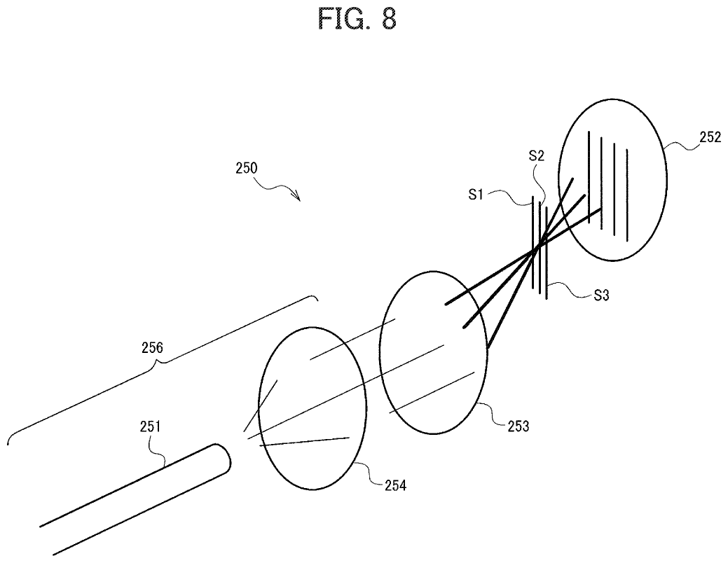

FIG. 8 illustrates a first detailed configuration example of the pattern light projection section 250. The pattern light projection section 250 includes an incident section 256, a DOE 253, and the slit section 252. The incident section 256 causes parallel lights including components with wavelengths λ1 to λ3 to be incident on the DOE 253. The slit section 252, on which a light emitted from the DOE 253 is incident, projects the pattern lights PT1 to PT3 with the wavelengths λ1 to λ3 onto the subject 5.

In accordance with the present embodiment, the pattern light source 150, which is a laser light source, is arranged in the control device 100, and only a simple optical system including the DOE 253 and the like is arranged at the leading end of the scope 200. This can reduce a diameter of the scope 200, and allows the laser light source to project the pattern lights PT1 to PT3 at high luminance To perform distance-measurement of the moving subject such as the living body with high accuracy, emission time of the pattern lights PT1 to PT3 needs to be made as short as possible, and using the laser light source enables reduction of emission time. The laser light source has an element that is larger than a light-emitting diode or the like. However, the configuration using the DOE 253 and the like allows the laser light source to be arranged in the control device 100 and can reduce the diameter of the scope 200. Since there is no need for arranging a heat generation source such as the light-emitting diode at the leading end of the scope 200, it is possible to prevent unnecessary heat generation at the leading end of the scope 200.

Note that in the ToF system, since an image sensor dedicated to ToF is arranged aside from the image sensor that captures the observation image, it is difficult to reduce the diameter. The present embodiment enables the reduction of the diameter as described above.

Details of the configuration illustrated in FIG. 8 are described below. The incident section 256 includes the optical fiber 251 that guides laser lights, and a collimate lens 254 that makes lights emitted from an optical fiber 251 parallel lights. The laser lights with wavelengths λ1 to λ3 are guided by one optical fiber 251, and diffused from an exit end of the optical fiber 251. The collimate lens 254 makes the diffused laser lights parallel lights.

The DOE 253 converges components with wavelengths λ1 to λ3 included in the parallel lights to a first linear light LL1 to a third linear light LL3 at mutually different positions. The slit section 252 has a plurality of mutually parallel slits. The linear lights LL1 to LL3 pass through the plurality of slits, whereby the pattern lights PT1 to PT3 are projected onto the subject.

The linear lights LL1 to LL3 function as virtual light sources that emit lights toward the slit section 252, and correspond to the light sources S1 to S3 illustrated in FIG. 1. Each linear light is parallel to the slits of the slit section 252. The linear lights LL1 to LL3 are arranged at different positions in the direction that is parallel to the plane of the slit section 252 and straight to the slits.

The DOE 253 is an optical element that controls an emitted light to have a specific shape by utilizing a diffraction phenomenon. The specific shape is determined depending on a fine structure of the DOE 253, and designing the fine structure enables obtaining of a light having a desired shape. In the present embodiment, the DOE 253 converges m-th order diffracted lights of incident parallel lights to linear lights with a predetermined focal length. A position at which the m-th order diffracted lights are converged is different depending on a wavelength. Since the incident lights includes components with the wavelengths λ1 to λ3, the m-th order diffracted lights with the respective wavelengths are converged as the linear lights LL1 to LL3 at mutually different positions. Note that m is an integer that is equal to or larger than 1. While the order is simply described herein as the m-th order, the m-th order may be either the +m-th order or the −m-th order.

The DOE 253 selectively converges the m-th order diffracted lights among zeroth, first, second, . . . order diffracted lights. That is, the m-th order diffracted lights emitted from the DOE 253 are greater in intensity than diffracted lights other than the m-th order diffracted lights. More specifically, the DOE 253 emits almost only the m-th order diffracted lights among the zeroth, first, second, . . . order diffracted lights.

The wavelengths λ1 to λ3 of the laser lights are, for example, at regular intervals. In this case, the DOE 253 converges the linear lights LL1 to LL3 so as to be at regular intervals. The “interval” mentioned herein is an interval in the direction that is parallel to the plane of the slit section 252 and straight to the slits. With the arrangement of the linear lights LL1 to LL3 at regular intervals, phases of the pattern lights PT1 to PT3 are at regular intervals. This enables emission of the pattern lights that are appropriate for the structured light system. In a case where functional calculation described above with reference to the Expression (6) is used, the phases of the pattern lights PT1 to PT3 need to be at regular intervals. Note that the wavelengths λ1 to λ3 of the laser lights may be at irregular intervals, and the linear lights LL1 to LL3 converged by the DOE 253 may be at irregular intervals. Even in this case, as described above with reference to the Expression (5), using the LUT enables conversion of the pattern image into the distance.

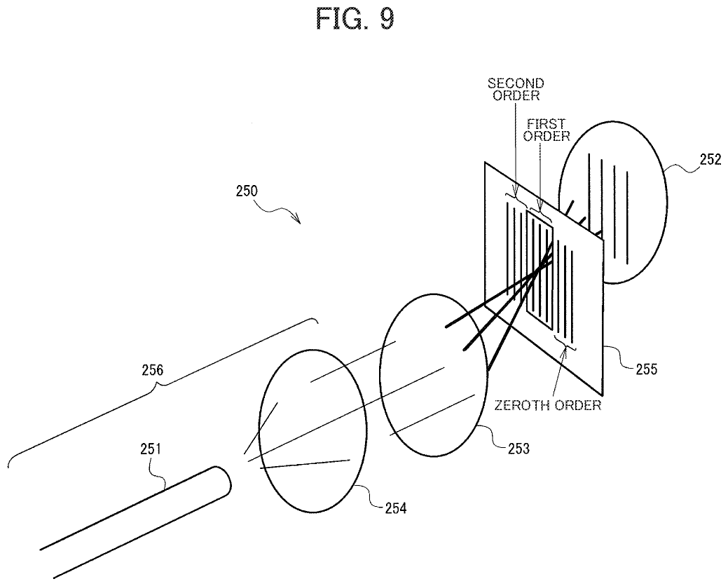

FIG. 9 illustrates a second detailed configuration example of the pattern light projection section 250. In FIG. 9, the pattern light projection section 250 further includes a mask section 255. Note that a constituent element that has been already described with reference to FIG. 8 is denoted by an identical reference sign, and a description thereof is omitted as appropriate.

The mask section 255 is arranged between the DOE 253 and the slit section 252. The mask section 255 causes the linear lights LL1 to LL3 converged from the m-th order diffracted lights to pass therethrough, and masks the diffracted lights other than the m-th order diffracted lights. FIG. 9 illustrates an example in which the mask section 255 causes the linear lights LL1 to LL3 converged from the first order diffracted lights to pass therethrough, and masks diffracted lights other than the first order diffracted lights, such as the zeroth order diffracted lights and the second order diffracted lights. The mask section 255 is a plate-like member that is parallel to the slit section 252, and apertures are arranged in the plate-like member. The mask section 255 is arranged so that the linear lights LL1 to LL3 pass through the apertures.

The DOE 253 selectively converges the m-th order diffracted lights, but lights emitted from the DOE 253 include diffracted lights other than the m-th order diffracted lights. When the diffracted lights other than the m-th order diffracted lights pass through the slit section 252, unnecessary pattern lights other than the pattern lights PT1 to PT3 that are originally intended are mixed in, and there is a possibility for reduction in accuracy of distance-measurement. In accordance with the present embodiment, the arrangement of the mask section 255 causes the diffracted lights other than the m-th order diffracted lights to be masked, thereby causes only the pattern lights PT1 to PT3 that are originally intended to be projected, and enables the distance-measurement with high accuracy.

In FIG. 6, the processing section 110 may perform AI processing to generate diagnosis support information. In this case, the storage section 120 stores a trained model that has been trained to generate the diagnosis support information, and the processing section 110 executes AI processing using the trained model to generate the diagnosis support information. Contents of the AI processing will be described below.

When the region of interest is designated in the observation image, the processing section 110 acquires distance information of the subject, and measures a length, height, or the like of the region of interest based on the distance information. The region of interest is designated by, for example, a user operation. Alternatively, the processing section 110 may perform AI image recognition on the observation image to detect the region of interest, and thereby designate the region of interest. The processing section 110 generates the diagnosis support information with the acquired length, height or the like of the region of interest and the observation image serving as input to the AI processing. The diagnosis support information is information for estimating, for example, whether or not there is a lesion, a type of the lesion, a degree of malignancy of the lesion, a shape of the lesion, and the like. The image output section 114 of the processing section 110 causes the display section 300 to display the diagnosis support information together with the observation image.

The processing section 110 may perform post-processing to generate the diagnosis support information. That is, the storage section 120 stores the observation image and the pattern image, and the processing section 110 may use the observation image and the pattern image stored in the storage section 120 to execute the AI processing.

Although the embodiments to which the present disclosure is applied and the modifications thereof have been described in detail above, the present disclosure is not limited to the embodiments and the modifications thereof, and various modifications and variations in components may be made in implementation without departing from the spirit and scope of the present disclosure. The plurality of elements disclosed in the embodiments and the modifications described above may be combined as appropriate to implement the present disclosure in various ways. For example, some of all the elements described in the embodiments and the modifications may be deleted. Furthermore, elements in different embodiments and modifications may be combined as appropriate. Thus, various modifications and applications can be made without departing from the spirit and scope of the present disclosure. Any term cited with a different term having a broader meaning or the same meaning at least once in the specification and the drawings can be replaced by the different term in any place in the specification and the drawings.

What is claimed is:

1. An endoscope system comprising:
a light source that emits lights with first to n-th wavelengths;
a lens that makes the lights with the first to n-th wavelengths parallel lights;
a diffractive optical element (DOE) that converges components of the lights with the first to n-th wavelengths, the components being included in the parallel lights, into first to n-th linear lights at mutually different positions;
a slit that projects, onto a subject, first to n-th pattern lights based on the first to n-th linear lights;
an imager that captures, as an one-frame image, an image of the subject onto which the first to n-th pattern lights are projected; and
a processor being configured to calculate a distance to the subject or a shape of the subject based on the image captured by the imager.

2. The endoscope system as defined in claim 1, wherein the lens, the DOE, and the slit simultaneously project the first to n-th pattern lights onto the subject in a frame in which the one-frame image is captured.

3. The endoscope system as defined in claim 1, further comprising:
a light source for observation that emits illumination light for observation, wherein
in a first frame, the light source for observation emits the illumination light for observation, and the imager captures an image of the subject illuminated with the illumination light for observation, and
in a second frame different from the first frame, the lens, the DOE, and the slit project the first to n-th pattern lights onto the subject, and the imager captures the image of the subject onto which the first to n-th pattern lights are projected.

4. The endoscope system as defined in claim 3,
wherein the processor
generates an observation image based on the image captured by the imager in the first frame,
calculates the distance or the shape based on the image captured by the imager in the second frame, and
performs artificial intelligence (AI) processing based on the observation image and the distance or the shape to make determination regarding detection of presence of a region of interest or discrimination of a state.

5. The endoscope system as defined in claim 3,
wherein the processor
generates an observation image based on the image captured by the imager in the first frame, and causes a display to display the observation image,
calculates the distance or the shape based on the image captured by the imager in the second frame as background processing of display of the observation image, and
adds information based on the distance or the shape to the observation image and causes the display to display the observation image.

6. The endoscope system as defined in claim 1, wherein wavelengths of the first to n-th pattern lights belong to a range of 460 nm or more and 700 nm or less.

7. The endoscope system as defined in claim 6, wherein the wavelengths of the first to n-th pattern lights belong to a range of 460 nm or more and 520 nm or less.

8. The endoscope system as defined in claim 1, wherein
the imager includes an image sensor having first to n-th color pixels that receive lights in first to n-th colors, respectively,
when wavelengths of the first to n-th pattern lights are first to n-th wavelengths, and sensitivity in the i-th color pixel (i is an integer that is equal to or larger than 1 and equal to or smaller than n) is aᵢⱼ, and
the processor extracts an image of the subject when each pattern light of the first to n-th pattern lights is projected based on the sensitivity aᵢⱼ , and intensity values of the first to n-th colors in the one-frame image, and calculates the distance to the subject or the shape of the subject from a phase based on the image of the subject when each pattern light is projected.

9. The endoscope system as defined in claim 8, wherein
n=3,
the first to n-th colors are red (R), green (G), and blue (B),
each row vector of a matrix A whose element is aᵢⱼ is a linearly independent vector, and
the processor calculates the following expression on an intensity value pᵢ in the i-th color at each position of the one-frame image to determine an intensity value qⱼ at each position of an image of the subject onto which the j-th pattern light is projected, and calculates the distance to the subject or the shape of the subject from a phase based on the intensity value qᵢ.
qⱼ=(A⁻¹)ⱼᵢpᵢ

10. The endoscope system as defined in claim 1, wherein the processor calculates .a gradient of a region of interest, which is a target of calculation of the shape, based on the distance to a periphery of the region of interest, and performs gradient correction on the shape of the region of interest based on the gradient.

11. The endoscope system as defined in claim 1, wherein
the DOE emits m-th order diffracted lights (m is an integer that is equal to or larger than 1) of the components with the first to n-th wavelengths, the components being included in the parallel lights, and
the m-th order diffracted lights are greater in intensity than diffracted lights other than the m-th order diffracted lights.

12. The endoscope system as defined in claim 1, further comprising a mask arranged between the DOE and the slit, wherein
the DOE emits m-th order diffracted lights of the components with the first to n-th wavelengths, the components being included in the parallel lights,
the m-th order diffracted lights are greater in intensity than diffracted lights other than the m-th order diffracted lights. and
the mask causes the first to n-th linear lights converged from the m-th order diffracted lights to pass therethrough, and masks diffracted lights other than the m-th order diffracted lights.

13. The endoscope system as defined in claim 1, wherein the first to n-th wavelengths are at regular intervals.

14. The endoscope system as defined in claim 1, further comprising an optical fiber, wherein
the light source emits laser lights with first to n-th wavelengths,
the optical fiber guides the laser lights, and
the lens is a collimate lens that makes lights emitted from the optical fiber the parallel lights.

15. An endoscope comprising:
a lens that makes lights with first to n-th wavelengths parallel lights;
a diffractive optical element (DOE) that converges components of the lights with the first to n-th wavelengths, the components being included in the parallel lights, into first to n-th linear lights at mutually different positions;
a slit that projects, onto a subject, first to n-th pattern lights based on the first to n-th linear lights; and
an imager that captures, as one-frame image, an image of the subject onto which the first to n-th pattern lights are projected.

16. A distance calculation method comprising:
a light source emitting lights with first to n-th wavelengths;
a lens making the lights with the first to n-th wavelengths parallel lights;
a diffractive optical element (DOE) converging components of the lights with the first to n-th wavelengths, the components being included in the parallel lights, into first to n-th linear lights at mutually different positions;
a slit projecting, onto a subject, first to n-th pattern lights based on the first to n-th linear lights;
an imager capturing, as one-frame image, an image of the subject onto which the first to n-th pattern lights are projected; and
a processor calculating a distance to the subject or a shape of the subject based on the image captured by the imager.
